(19) United States
(12) Patent Application Publication
(10) Pub. No.: US 2011/0287096 A1
(43) Pub. Date: Nov. 24, 2011

(54) MODIFIED GASTRORETENTIVE DRUG DELIVERY SYSTEM FOR AMINE DRUGS

(75) Inventors: Sudhir GORUKANTI, Tuxedo Park, NY (US); Yanming Zu, Highland Mills, NY (US); Phanidhara Kotamraj, Westwood, NJ (US); Karunakar Neelam, New Milford, NJ (US); Salah U. Ahmed, New City, NY (US)

(73) Assignee: ABON PHARMACEUTICALS, LLC, Northvale, NJ (US)

(21) Appl. No.: 13/110,591

(22) Filed: May 18, 2011

Related U.S. Application Data
(60) Provisional application No. 61/345,826, filed on May 18, 2010.

(51) Int. Cl. A61K 9/62; A61K 31/506; A61K 9/58; A61K 9/56; A61K 9/26; A61K 9/54

(52) U.S. Cl. 424/461; 424/469; 424/458; 424/462; 424/459; 514/252.19

(57) ABSTRACT

Oral dosage forms for basic amine drugs, the dosage forms having a gastro-retentive component and a non gastro-retentive component. These dosage forms are capable of providing both IR and SR release rates for these drugs. In addition, they provide for release of the drug in the stomach and/or intestine of a mammal to which such dosage forms are administered. Such dosage forms include tablets and capsules. Such dosage forms provide improved bioavailability of otherwise poorly bioavailable basic amine drugs.

### CROSS REFERENCE TO RELATED APPLICATIONS

This application claims priority from U.S. Provisional Application Ser. No. 61/345,826 filed on May 18, 2010 and incorporates herein the contents thereof in their entirety.

### FIELD OF THE INVENTION

The present invention relates to oral pharmaceutical delivery systems and dosage forms.

### BACKGROUND OF THE INVENTION

The delivery of basic drugs, or active pharmaceutical ingredients, to patients by oral administration for absorption in the gastrointestinal tract presents challenges, as such drugs have a very low solubility at the pH found in the small intestine, and thus do not dissolve well in the intestine. However, the drug must dissolve to be absorbed by the body. When basic drugs are delivered in a standard immediate release oral dosage form, some of the drug may dissolve in the stomach and be absorbed, but unabsorbed drug will not dissolve in the intestine or will precipitate in the intestine, and eventually will be excreted as solid waste. In a sustained or delayed oral release dosage form, much or all of the drug may remain in the dosage form until it reaches the intestine, and again much of the drug will not dissolve and be excreted as waste.

Currently existing dosage forms are limited in their ability to deliver basic active compounds in an effective manner. For example, such dosage forms are incapable of improving the solubility of basic drugs in the intestinal pH, and cannot deliver basic drugs throughout the intestine.

One possible solution to this problem is to administer the basic drug in an oral dosage form that is retained in the stomach for a significant period of time (a gastro-retentive dosage form), where the drug can be released into an acid pH of the stomach, and dissolve there before moving into the intestine. An example of a gastro-retentive dosage form is one that floats after it is administered, and thereby remains in the stomach until the dosage form erodes. However, existing floating dosage forms (1) after administration do not quickly float, (2) do not have adequate buoyancy because of insufficient gas entrapment in the matrix, (3) have poor matrix integrity which minimizes their effectiveness in controlling release of drugs for extended periods of time (4) tend to create mucoadhesion which may contribute to irritation and ulcers in the stomach, and (5) require additional layers or coating to entrap gas and create buoyancy.

The present invention unexpectedly overcomes these limitations.

### SUMMARY OF THE INVENTION

The present invention provides gastro-retentive oral dosage forms which exhibit both immediate release (IR) and controlled, sustained or extended release (CR, SR or ER) properties. These dosage forms comprise (a) a gastro-retentive component that is capable of being retained in the stomach and releasing drug while the dosage form remains in the stomach and (b) a non gastro-retentive component that is not designed to be retained in the stomach and comprises an acidifier that can create an acid microenvironment that improves dissolution of a basic amine drug in the intestine. Having both gastro-retentive and non gastro-retentive components and the use of SR polymers in one or more of the components in the dosage form provides a release profile that allows immediate release of the drug (the release can begin soon after the dosage form comes into contact with gastric fluids), but also allows drug release to be sustained over a period of time in both stomach and intestine.

In one embodiment of the present invention such oral dosage forms release an IR component of an active ingredient in the stomach of a mammal and deliver a CR, SR or ER component of an active ingredient in the stomach and/or intestinal tract of the mammal.

In another embodiment of the present invention such oral dosage forms release a CR, SR or ER component of an active ingredient in the stomach of a mammal and deliver an IR component of an active ingredient in the stomach and/or intestinal tract of the mammal. In another embodiment of the present invention the active component includes drugs that contain an amine group and exhibit reasonable stability at hydronium ion concentrations above 10e⁻⁷ and/or exhibit pH-dependent solubility.

A further embodiment of the present invention provides drug delivery systems for oral dosage forms that include (1) one or more hydrophilic insoluble polymers, (2) one or more acidifiers, (3) one or more carbonic compounds, (4) one or more water insoluble hydrophilic fluid penetrating agents, and (5) active components in both IR and CR, SR or ER forms.

### BRIEF DESCRIPTION OF THE DRAWINGS

FIG. 1 illustrates a representative gastro-retentive tablet which includes both IR and SR components.

FIG. 2 illustrates a representative gastro-retentive capsule which includes both IR and SR components.

FIG. 3 illustrates a representative gastro-retentive capsule which includes IR mini-tablets and SR beads.

FIGS. 4A-C illustrate a representative process for formulating a gastro-retentive tablet having a non-gastroretentive component, and having both IR and SR properties.

FIGS. 5A-C illustrate a representative process for formulating a capsule having both gastro-retentive and non gastro-retentive components, the capsule having IR and SR properties.

FIGS. 6A-D illustrate a representative process for formulating another representative capsule having both gastro-retentive and non-gastroretentive components and IR and SR properties.

### DETAILED DESCRIPTION OF THE INVENTION

The present invention provides oral dosage forms which comprise gastro-retentive and non gastro-retentive components and exhibit both immediate release (IR) and controlled, sustained or extended release (CR, SR or ER) properties. Such dosage forms include, but are not limited to, tablets and capsules.

Such dosage forms may be used to deliver the immediate release portion of the drug in the stomach while maintaining the integrity of the dosage form. After administration the gastro-retentive component of such dosage forms may be retained in the stomach for a prolonged period while the extended release portion of the drug may be delivered in the stomach and/or intestine. In addition, the non gastro-retentive component, with acidifier, releases drug in the intestine. By having a component retained in the stomach for prolonged periods, and another component with acidifier that provides release in the intestine, such dosage forms will increase the bioavailability of basic drugs and drugs that exhibit site specific absorption in the gastrointenal tract. Such dosage forms will also deliver the drug in the intestine without precipitation of the drug out of solution. Further advantages include rapid hydration of the gastro-retentive component matrix, excellent buoyancy and good matrix integrity upon hydration.

Unless otherwise noted, as used herein CR, SR and ER are used interchangeably to refer to release of an active component at a rate which is controlled, delayed or extended in comparison to immediate release.

Embodiments of the present invention are particularly well-suited for oral dosage forms in which the active compound contains an amine group (i.e., a group containing a nitrogen moiety). In certain embodiments the active compound includes drugs that are basic in nature. In further embodiments the active compound also may be stable at an acidic pH.

Examples of basic amine drugs include, but are not limited to, Alfuzosin, Amiodarone, Bupivacaine, Carvedilol, Celiprolol, Chlorprothixene, Cyproheptadine, Dasatinib, Desipramine, Dipyridamole, Disopyramide, Donepezil, Fendiline, Haloperidol, Hydralazine, Imatinib, Etoposide, Lidocaine, Maprotiline, Mifepristone, Nilotinib, Orphenadrine, Paliperidone, Pramoxine, Procyclidine, Promethazine, Propafenone, Silodosin, Terazosin, Thioridazine, Trihexyphenidyl, Trimethoprim, Verapamil and the like. Such active compounds may include, but are not limited to, those in the following general categories of active agents: acromegaly agents, alcohol abuse preparations, analgesics, antiasthmatics, anticancer agents, anticoagulants and antithrombic agents, anticonvulsants, antidiabetic agents, antiemetics, antiglaucoma agents, antihistamines, anti-infective agents, anti-Parkinson's agents, antirheumatic agents, platelet agents, antispasmodics and anticholinergic agents, antitussives, carbonic anhydrase inhibitors, cardiovascular agents, cholinesterase inhibitors, CNS stimulants, contraceptives, cystic fibrosis management agents, dopamine receptor agonists, endometriosis management agents, erectile dysfunction agents, fertility agents, gastrointestinal agents, immunomodulators and immunosupressives, Alzheimer's disease agents, migraine preparations, muscle relaxants, nucleoside analogues, osteoporosis management agents, parasympathomimetics, psychotherapeutic agents, sedatives, hypnotics and tranquilizers, agents for treatment of skin ailments, steroids and hormones.

In another embodiment of the present invention the active compound exhibits pH-dependent solubility.

In certain embodiments the active compound to excipient ratio by weight may be from about 1:0.25 to about 0.1:100, or preferably from about 1:4 to about 1:50.

Swelling hydrophilic insoluble polymers according to the present invention are typically easily dispersible in water with transparent colloidal or opaque gel-type mass formation upon hydration. Such hydrophilic, insoluble polymers may include, but are not limited to: acrylic acid and its methacrylic acid polymers, alginates, carboxy methyl cellulose and its sodium and calcium salts, guar gum, gum arabic, hydroxypropylcellulose, hydroxypropylmethylcellulose, methyl hydroxyethyl cellose, polyethylene oxide, polyvinyl pyrrolidone, pregelatinized starch, xanthan gum and combinations thereof.

In one embodiment of the invention the one or more hydrophilic insoluble polymers may be present in an amount greater than about 5% by weight of the total dosage form. In certain embodiments the one or more hydrophilic insoluble polymers may be present in a range from about 5% to about 95%, or preferably about 10% to about 30%.

Acidifiers according to the present invention may include low molecular weight acidic substances, high molecular weight acidic substances, and combinations of low and high molecular weight substances. Low molecular weight acidic substances include, but are not limited to, acids, such as acetic acid, ascorbic acid, citric acid, fumaric acid, malic acid, maleic acid, malonic acid, aspartic acid, tartaric acid, sorbic acid, succinic acid, glutamic acid, hydrochloride, hydrochloric acid, phosphoric acid, carbonic acid and mixtures thereof. Low molecular weight acidic substances may also include acid salts, such as sodium or potassium hydrogen sulphate, betaine hydrochloride, sodium or potassium salts of tartaric acid, sorbic acid or citric acid and combinations thereof.

High molecular weight acidic substances include but are not limited to hydroxypropyl methylcellulose acetate succinate, methacrylic acid methyl methacrylate copolymer, methacrylic acid ethyl acrylate copolymer and combinations thereof.

One or more acidifiers may be present in a mole equivalent ratio to the amine drug of about 0.1:1 to about 50:0.1, preferably about 0.5:1 to about 20:1.

Carbonic compounds according to the present invention may include but are not limited to calcium carbonate, dihydroxy aluminum sodium carbonate, magnesium carbonate, potassium bicarbonate, potassium carbonate, sodium bicarbonate, sodium carbonate, sodium carbonate monohydrate, zinc carbonate, and combinations thereof.

One or more carbonic compounds may be included in amounts ranging from about 10:0.1 to about 0.1:50, preferably about 5:0.1 to about 0.1:20, mole basic equivalents relative to the acidifier.

One or more water insoluble fluid penetrating agents also may be included. Such fluid penetrating agents include but are not limited to sodium starch glycolate (Primojel®, Explotab®, Glycolys®, Vivastar®, Explosol®), croscarmellose sodium (Ac-di-sol®, Vivasol®, Explosel®), methylcellulose, carboxy methyl cellulose, microcrystalline cellulose (Avicel PH 101, PH 102, PH 105), ion exchange resins (Amberlite IRP 88) and combinations thereof.

In certain embodiments the sum of the weight % of hydrophilic insoluble polymers and the water insoluble fluid penetrating agent ranges from about 10 to about 90% of the weight of the dosage form, preferably from about 20% to about 60% of the weight of the dosage form, of which the ratio of the former to the latter may be from about 1:10 to about 10:1, preferably about 1:5 to about 5:1.

In certain embodiments tablets or capsules according to the present invention also may include binders, diluents, lubricants, glidants, surfactants, permeation enhancers and/or organic acidifiers. These excipients are known in the art.

Examples of binders are hydroxylpropyl cellulose, hydroxypropylmethyl cellulose and polyvinyl pyrrolidone (e.g., Povidone K30 and K90). Examples of diluents are dicalcium phosphate, calcium phosphate, starch, microcrystalline cellulose NF (e.g., Avicel® pH 101 or other grades), more preferably-starch and/or microcrystalline cellulose, and most preferably microcrystalline cellulose). It is preferred that the diluent lactose not be used in the same component as the basic amine drug, as it may cause a Maillard reaction. Lubricants may include, but are not limited to magnesium stearate, sodium stearyl fumarate, aluminum stearate, hydrogenated vegetable oil or stearic acid, NF. Glidants (flow enhancer) may include, but are not limited to, silicon dioxide NF, talc, and most preferably colloidal silicon dioxide (e.g., Cab-O-Sil, Aerosil 200). Surfactants, and permeation enhancers may include but are not limited to PEG fatty acid mono and di esters (C₆ to C₁₈), poloxamers (Lutrol F68, Lutrol micro 68, Lutrol F127 NF, Lutrol micro 127), polyvinyl caprolatam-polyvinyl acetate polyethylene glycol (Soluplus), propylene glycol fatty acid esters, sodium laurylsulfate, sugar ester surfactant, Vitamin E TPGS and combinations thereof.

Dosage forms according to the present invention may provide a ratio of IR active agent(s) to CR, SR or ER active agent(s) from about 1:10 to 10:1. In certain embodiments the ratio may be from about 1:5 to about 5:1 and in other embodiments from about 1:2 to about 2:1.

In certain embodiments the oral dosage form may be in the form of a tablet. The tablet may be a gas-generating floating matrix with both IR and SR properties. The drug embedded homogenously within the matrix may contribute to an immediate release of the active drug. The SR portion of the tablets may be made of drug-loaded acidified pellets coated with sustained releasing agents with enteric or non-enteric properties. The tablet may retain its integrity over a prolonged period of time as a floating matrix in the stomach. Once the tablet loses its gastric retention property, the drug-loaded acidified SR pellets may pass into the small intestine continuing to release the active drug despite the higher pH in the small intestine. This may be achieved based on the micro-acidic environment of the drug maintained within the membrane of SR pellets.

In certain embodiments the oral dosage form may be in the form of a hard gelatin capsule containing two or more types of drug-containing particles. Such capsules may include IR particles in the form of granules/powder/beads/micro tablets. Such capsules also may include gastro-retentive SR particles in the form of micro tablets or beads. The SR particles may be acidified micro-tablets or beads capable of floating, coated with sustained releasing agents with enteric or non-enteric properties. The floating SR coated acidified micro-tablets or beads may release the active drug in a controlled manner in the stomach and continue to release the drug in the lower part of the GI tract, due to the micro-acidic environment of the drug maintained within the membrane of SR particles.

In certain embodiments the oral dosage form may be in the form of a hard gelatin capsule with both IR and SR properties. Such capsules may include IR particles in the form of floating micro tablet. The drug embedded homogenously within the micro tablet matrix may contribute to an immediate drug release. Such capsules also may include gastro-retentive SR particles in the form of micro-tablets or beads. The SR particles may be acidified micro tablets or beads capable of floating, coated with sustained releasing agents with enteric or non-enteric properties. The floating SR coated acidified micro tablets or beads release drug in a controlled manner in the stomach and continue drug release in the lower part of the GI tract, due to the micro-acidic environment of the drug maintained within the membrane of SR particles. In addition, such capsules also may include SR particles in the form of beads. The starting beads may comprise acidifiers such as tartaric acid pellets. The acidifier seeds may be coated with active drug followed by the functional SR coat, providing for the release of active drug in the lower part of the GI tract due to the micro-acidic environment of the drug maintained within the membrane of the SR coat.

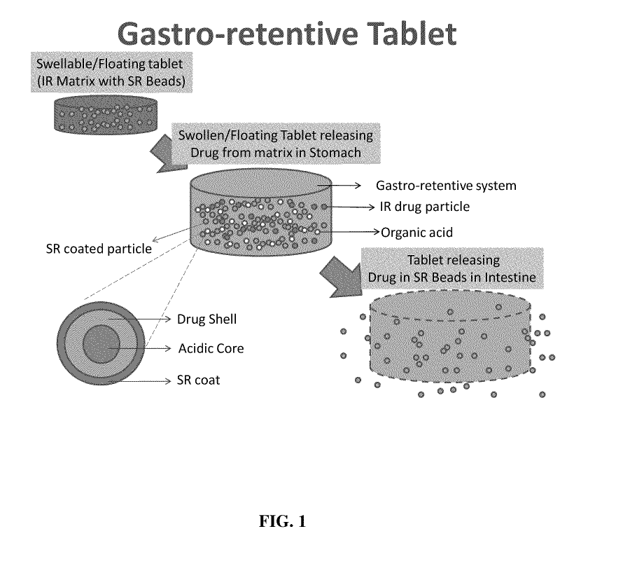

As shown in FIG. 1, a gastro-retentive tablet according to the present invention may contain both IR and SR particles. When such tablets are administered to a mammal they may release the IR particles in the stomach of the mammal and release the SR particles in the intestine. The SR particles may comprise an SR coating, an active ingredient shell and an acidic core. The acidic core may include one or more of the acidifiers mentioned above.

SR polymers may include but are not limited to cellulose acetate phthalates copolymers of methacrylic acid, methacrylate and methyl methacrylate (Eudragit FS 30D), ethyl cellulose, hydrogenated glyceryl esters of fatty acids (or of castro wax, carnauba wax etc.), Kollicoat SR methacrylic acid ethyl acrylate copolymer (Eudragit L30D-55, Kollicoat MAE 30DP), methacrylic acid copolymers with trimethyl ammoniumethyl methacrylate (Eudragit RL 30D, RL PO, RL 100, RS 30D, RS PO, RS 100), methcrylic acid-ethyl acrylate copolymer (1:1) (Eudragit L100, Kollicoat MAE 100P; Eudragit S100), neutral polymer of methacrylate (Eudragit ne 30D), and combinations thereof.

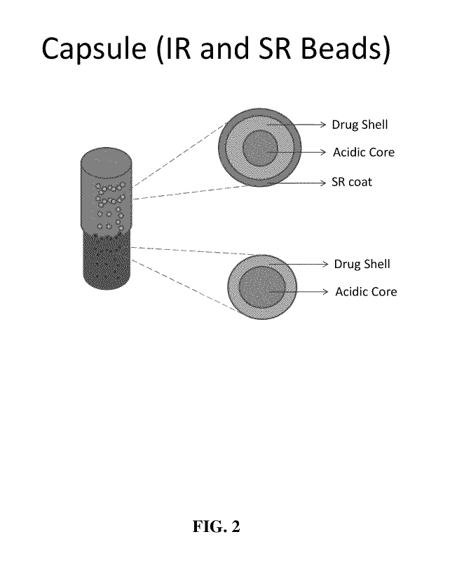

As shown in FIG. 2, a gastro-retentive capsule according to the present invention may contain both IR and SR beads. When such tablets are administered to a mammal they may release the IR particles in the stomach of the mammal and release the SR particles in the stomach and/or intestine. The IR particles may include a drug shell and an acidic core. The SR particles may comprise an SR coating, an active ingredient shell and an acidic core.

SR polymers may include those listed above with respect to tablet dosage forms. The acidic core may comprise one or more of the acidifiers mentioned above.

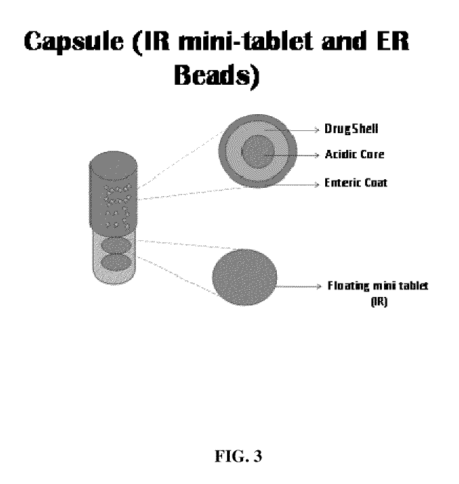

As shown in FIG. 3, capsules according to the present invention may contain both one or more floating IR mini-tablets and ER beads. The ER beads in such capsules may include a drug shell, acidic core and enteric (pH dependent) coat as described above.

The active ingredient of the gastro-retentive component of the dosage form may be the same as or different from the active ingredient in the non gastro-retentive component of the dosage form. In addition, the active ingredient of the IR portion of the dosage form may be the same as or different from the SR portion of the dosage form.

### Example 1

A tablet as shown in Table 1 below may be administered to a mammal to deliver a portion of an active ingredient in the stomach of the mammal and a portion of an active ingredient in the stomach and/or intestinal tract of the mammal.

TABLE 1
Representative Tablet

| | | |
|---|---|---|
| Ingredient | Function | Weight % |

Gastro-retentive component of the tablet:

| | | |
|---|---|---|
| Dasatinib | Basic Amine Drug | 25 |
| SwelStar MX-1 | Swelling Hydrophilic | 20 |
| | Insoluble Polymer | |
| Tartaric acid | Acidifier | 6 |
| Sodium bicarbonate | Carbonic compound | 6 |
| Sodium starch glycolate | Water Insoluble Fluid | 2.25 |
| | Penetrating Agent | |
| Cab-osil | Glidant | 0.5 |
| Magnesium Stearate | Lubricant | 0.25 |
| Gastro-retentive Component weight | | 60 |

Non gastro-retentive Component (Beads):

| | | |
|---|---|---|
| Dasatinib | Basic Amine Drug | 25 |
| Tartaric acid bead | Acidifier/solubulizer | 8.75 |
| Hydroxypropyl cellulose | Binder | 3.125 |
| Ethyl cellulose, EC10 | SR polymer | 1.625 |
| Eudragit, S100 | SR Polymer | 1.25 |
| Isopropyl alcohol* | Solvent | (21.875) |
| Water* | Solvent | (9.375) |
| Non gastro-retentive Component | | 40 |
| (Beads) weight | | |
| Total tablet weight | | 100 |

*Not a part of the finished product; Evaporated during the process.

The gastro-retentive component matrix blend may be manufactured by mixing the active and other excipients in a blender until uniformly mixed. If the active exhibits poor flow and/or low bulk density, the blend may be roller-compacted to achieve free-flowing denser granules.

The non gastro-retentive component coated beads may be manufactured in a fluid bed dryer by coating the tartaric acid beads with protective layer dispersion, drug layer dispersion and SR layer coating dispersion. Alternatively, the coated beads also may be manufactured by extrusion spheronization of drug, acid and binder to form pellets. These pellets may be coated with an SR layer coating to get SR coated beads.

The matrix blend and SR beads may be mixed in a blender until uniformly mixed. This blend may be compressed into a tablet dosage form.

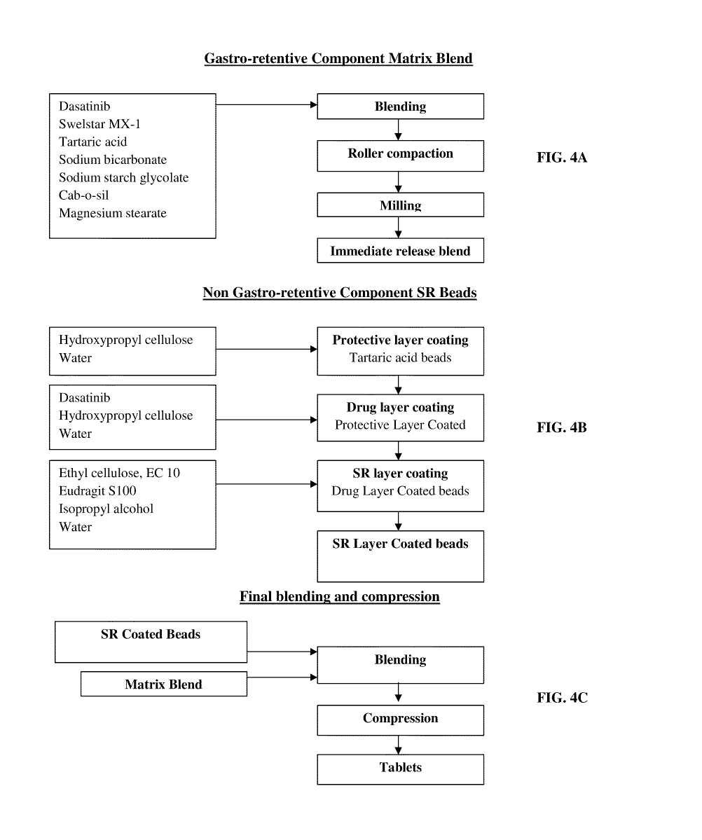

FIGS. 4A-4C provide a flow chart illustrating such a process.

### Example 2

A capsule as shown in Table 2 below may be administered to a mammal to deliver a portion of an active ingredient in the stomach of the mammal and a portion of an active ingredient in the stomach and/or intestinal tract of the mammal.

TABLE 2
Representative Capsule

| | | |
|---|---|---|
| Ingredient | Function | Weight % |

Non gastro-retentive component (micro tablet or granules) of the Capsule:

| | | |
|---|---|---|
| Dasatinib | Basic Amine Drug | 28.57 |
| Tartaric acid | Acidifier | 5.71 |
| Poloxamer, 188 | Surfactant | 0.29 |
| Microcrystalline cellulose | Diluent | 7.14 |
| (Avicel) | | |
| Magnesium Stearate | Lubricant | 0.29 |
| Non gastro-retentive | | 42.00 |
| Component weight | | |

Gastro-retentive (micro tablet or granules):

| | | |
|---|---|---|
| Dasatinib | Basic Amine Drug | 28.57 |
| SwelStar MX-1 | Swelling Hydrophilic | 11.43 |
| | Insoluble Polymer | |
| Tartaric acid | Acidifier | 6.07 |
| Sodium bicarbonate | Carbonic Compound | 6.07 |
| Sodium starch glycolate | Water Insoluble Fluid | 5.00 |
| | Penetrating Agent | |
| Cab-osil | Glidant | 0.50 |
| Magnesium Stearate | Lubricant | 0.36 |
| Gastro-retentive component | | 58.00 |
| weight | | |
| Total capsule fill weight | | 100 |

The non gastro-retentive component may be manufactured by blending drug and other excipients in a blender until uniformly mixed. If the drug exhibits poor flow and/or low bulk density, the powder blend may be roller-compacted to achieve free-flowing and denser granules. These granules may be milled and screened to achieve a free-flowing blend. This blend may be further compressed into micro tablets or may be filled as a powder for the immediate release portion of the capsule.

The gastro-retentive component may be a micro tablet manufactured by blending drug and other excipients in a blender until uniformly mixed and compressed in to tablets. If the drug exhibits poor flow and/or low bulk density, the powder blend may be roller-compacted to achieve free-flowing and denser granules. These granules may be milled and screened to achieve a free-flowing blend. This blend may be further compressed into micro tablets or may be filled as a powder for the immediate release portion of the capsule.

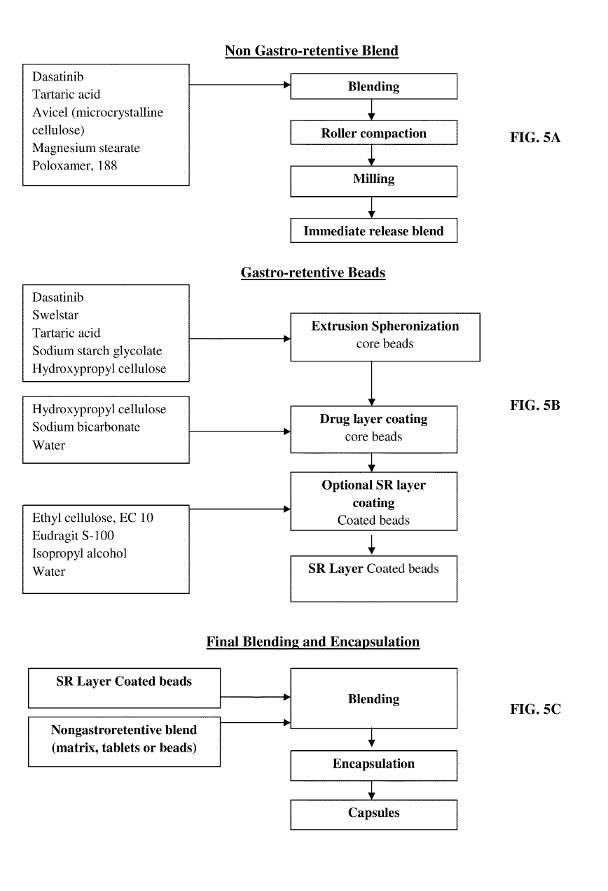

Alternatively, the gastro-retentive component, may include beads manufactured by extrusion spheronization of basic amine drug, acidifier, swelling hydrophilic insoluble polymer and binder to form pellets. These pellets may be coated with a protective layer coating and carbonic compound layer coating and, optionally, may be followed by a sustained release layer coating. This process is described in FIG. 5B.

The non gastro-retentive blend and/or micro-tablets and gastro-retentive micro tablets or beads may be used in capsules to achieve a combined immediate release and extended release capsule dosage form.

FIGS. 5A-5C provide a flow chart illustrating such a process, where the gastro-retentive component is a bead, instead of a micro-tablet.

### Example 3

A capsule as shown in Table 3 below may be administered to a mammal to deliver a portion of an active ingredient in the stomach of the mammal and a portion of an active ingredient in the stomach and/or intestinal tract of the mammal.

TABLE 3
Representative Capsule

| | | |
|---|---|---|
| Ingredient | Function | Weight % |

Gastro-retentive (Micro Tablets)

| | | |
|---|---|---|
| Dasatinib | Basic Amine Drug | 12.5 |
| SwelStar MX-1 | Swelling Hydrophilic | 10 |
| | Insoluble Polymer | |
| Tartaric acid | Acidifier | 3 |
| Sodium bicarbonate | Carbonic Compound | 3 |
| Sodium starch glycolate | Water Insoluble Fluid | 1.15 |
| | Penetrating Agent | |
| Cab-osil | Glidant | 0.25 |
| Magnesium Stearate | Lubricant | 0.1 |
| Weight | | 30 |

Gastro-retentive (Micro Beads)

| | | |
|---|---|---|
| Dasatinib | Basic Amine Drug | 10 |
| SwelStar MX-1 | Swelling Hydrophilic | 8 |
| | Insoluble Polymer | |
| Tartaric acid | Acidifier | 6 |
| Sodium bicarbonate | Carbonic Compound | 3 |
| Sodium starch glycolate | Water Insoluble Fluid | 1.5 |
| | Penetrating Agent | |
| Ethyl cellulose, EC10 | SR polymer | 1.2 |
| Dibutyl Sebacate | Plasticizer | 0.3 |
| Isopropyl alcohol/Water* | Solvent | |
| Weight | | 30 |

Non gastro-retentive Beads

| | | |
|---|---|---|
| Dasatinib | Basic Amine Drug | 16 |
| Tartaric acid pellets | Acidifier/solubulizer | 17.5 |
| Povidone K90 | Binder | 5 |
| Eudragit L100 55 | SR pH dependent polymer | 0.3 |
| Eudragit, S100 | SR pH dependent polymer | 0.3 |
| Ethyl cellulose, EC10 | SR pH independent polymer | 0.6 |
| Dibutyl Sebacate | Plasticizer | 0.3 |
| Isopropyl alcohol/Water* | Solvent | |
| Weight | | 40 |
| Total capsule fill weight | | 100 |

Gastro-retentive micro tablets/beads may be manufactured by extrusion spheronization or direct compression of drug, acid, swelling polymer and binder. These microtablets or beads may be further coated with a sustained release coating to obtain the gastro-retentive sustained releases micro tablets or beads.

In addition, another sustained release portion of the capsule may be provided by layering active drug onto tartaric acid pellets. The drug-loaded tartaric acid may be further coated with a functional sustained release coating to achieve an extended drug release in the higher pH environment of the lower GI tract. Gastro-retentive IR and SR microtablets/beads, along with SR beads may be filled in to a capsule to achieve gastro-retentive extended release capsule dosage form throughout the whole GI tract.

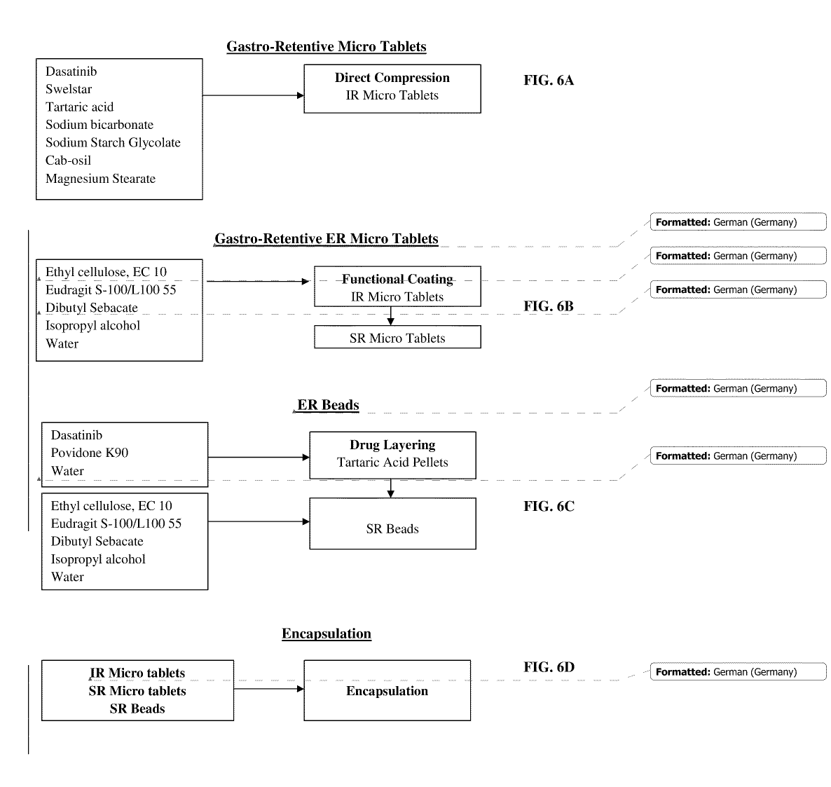

FIGS. 6A-6D provide a flow chart illustrating such a process.

What is claimed is:

1. An oral dosage form comprising,
(a) a gastrorentive component comprising a basic amine drug, an acidifier,
a carbonic compound, a hydrophilic insoluble polymer, and a water insoluble fluid penetrating agent; and
(b) a non gastro-retentive component comprising a basic amine drug, an acidifier and a sustained release polymer.

2. The oral dosage form of claim 1, wherein said gastro-retentive component provides release of said basic amine drug in the stomach, and said non gastro-retentive component provides sustained, delayed or extended release of said basic amine drug.

3. The oral dosage form of claim 1, wherein the hydrophilic insoluble polymer is selected from the group consisting of acrylic acid, methacrylic acid polymers, alginates, carboxy methyl cellulose and its sodium and calcium salts, guar gum, gum arabic, hydroxypropylcellulose, hydroxypropylmethylcellulose, methyl hydroxyethyl cellose, modified starch, polyethylene oxide, polyvinyl pyrrolidone, pregelatinized starch, xanthan gum, and combinations thereof.

4. The oral dosage form of claim 1, wherein the acidifier is selected from the group consisting of ascorbic acid, citric acid, fumaric acid, malic acid, maleic acid, malonic acid, aspartic acid, tartaric acid, sorbic acid, succinic acid, glutamic acid, hydrochloride, an acid salt, and combinations thereof.

5. The oral dosage form of claim 1, wherein the carbonic compound is selected from the group consisting of calcium carbonate, dihydroxy aluminum sodium carbonate, magnesium carbonate, potassium bicarbonate, potassium carbonate, sodium bicarbonate, sodium carbonate, zinc carbonate, and combinations thereof.

6. The oral dosage form of claim 1, wherein the water insoluble fluid penetrating agent is selected from the group consisting of sodium starch glycolate, croscarmellose sodium, methylcellulose, carboxy methyl cellulose, microcrystalline cellulose, ion exchange resins, and combinations thereof.

7. The oral dosage form of claim 1, wherein the sustained release polymer is selected from the group consisting of cellulose acetate phthalates, copolymers of methacrylic acid, copolymers of methacrylate, copolymers of methyl methacrylate, ethyl cellulose, hydrogenated glyceryl esters of fatty acids, castro wax, carnauba wax, methacrylic acid ethyl acrylate copolymer, methacrylic acid copolymers with trimethyl ammoniumethyl methacrylate, methcrylic acid-ethyl acrylate copolymer (1:1), neutral polymers of methacrylate, and combinations thereof.

8. The oral dosage form of claim 1, wherein the basic amine drug in said gastro-retentive component is different from the basic amine drug in said non gastro-retentive component.

9. The oral dosage form of claim 1, wherein said insoluble hydrophilic insoluble polymer is pregelatinized starch, and said water insoluble fluid penetrating agent is sodium starch glycolate.

10. The oral dosage form of claim 9, wherein said sustained release polymer is a methacrylic acid copolymer.

11. An oral dosage form comprising,
(a) a gastro-retentive component comprising a basic amine drug, an acidifier, a carbonic compound, a hydrophilic insoluble polymer, and a water insoluble fluid penetrating agent; and
(b) a non gastro-retentive component comprising a basic amine drug and an acidifier.

12. The oral dosage form of claim 11, wherein said gastro-retentive component also comprises a sustained release polymer.

13. The oral dosage form of claim 12, wherein said gastro-retentive component provides sustained release of said basic amine drug and said non gastro-retentive component provides immediate release of said basic amine drug.

14. The oral dosage form of claim 12, wherein the sustained release polymer is selected from the group consisting of cellulose acetate phthalates, copolymers of methacrylic acid, copolymers of methacrylate, copolymers of methyl methacrylate, ethyl cellulose, hydrogenated glyceryl esters of fatty acids, castro wax, carnauba wax, methacrylic acid ethyl acrylate copolymer, methacrylic acid copolymers with trimethyl ammoniumethyl methacrylate, methcrylic acid-ethyl acrylate copolymer (1:1), neutral polymers of methacrylate, and combinations thereof.

15. The oral dosage form of claim 11, wherein the hydrophilic insoluble polymer is selected from the group consisting of acrylic acid, methacrylic acid polymers, alginates, carboxy methyl cellulose and its sodium and calcium salts, guar gum, gum arabic, hydroxypropylcellulose, hydroxypropylmethylcellulose, methyl hydroxyethyl cellose, modified starch, polyethylene oxide, polyvinyl pyrrolidone, pregelatinized starch, xanthan gum, and combinations thereof.

16. The oral dosage form of claim 11, wherein the acidifier is selected from the group consisting of ascorbic acid, citric acid, fumaric acid, malic acid, maleic acid, malonic acid, aspartic acid, tartaric acid, sorbic acid, succinic acid, glutamic acid, hydrochloride, an acid salt, and combinations thereof.

17. The oral dosage form of claim 11, wherein the carbonic compound is selected from the group consisting of calcium carbonate, dihydroxy aluminum sodium carbonate, magnesium carbonate, potassium bicarbonate, potassium carbonate, sodium bicarbonate, sodium carbonate, zinc carbonate, and combinations thereof.

18. The oral dosage form of claim 11, wherein the water insoluble fluid penetrating agent is selected from the group consisting of sodium starch glycolate, croscarmellose sodium, methylcellulose, carboxy methyl cellulose, microcrystalline cellulose, ion exchange resins, and combinations thereof.

19. The oral dosage form of claim 11, wherein the basic amine drug in said gastro-retentive component is different from the basic amine drug in said non gastro-retentive component.

20. The oral dosage form of claim 11, wherein said insoluble hydrophilic insoluble polymer is pregelatinized starch, and said water insoluble fluid penetrating agent is sodium starch glycolate.

21. The oral dosage form of claim 12, wherein said sustained release polymer is a methacrylic acid copolymer.

22. An oral dosage form comprising,
(a) a first gastrorentive component comprising a basic amine drug, an acidifier,
a carbonic compound, a hydrophilic insoluble polymer, and a water insoluble fluid penetrating agent;
(b) a second gastrorentive component comprising a basic amine drug, an acidifier, a carbonic compound, a hydrophilic insoluble polymer, a water insoluble fluid penetrating agent, and a sustained release polymer; and
(c) A non gastro-retentive component comprising a basic amine drug, an acidifier and a sustained release polymer.

23. The oral dosage form of claim 22, wherein said first gastro-retentive component provides immediate release of said basic amine drug, said second gastro-retentive component provides sustained release of said basic amine drug, and said non gastro-retentive component provides sustained release of said basic amine drug

24. The oral dosage form of claim 22, wherein the hydrophilic insoluble polymer is selected from the group consisting of acrylic acid, methacrylic acid polymers, alginates, carboxy methyl cellulose and its sodium and calcium salts, guar gum, gum arabic, hydroxypropylcellulose, hydroxypropylmethylcellulose, methyl hydroxyethyl cellose, modified starch, polyethylene oxide, polyvinyl pyrrolidone, pregelatinized starch, xanthan gum and combinations thereof.

25. The oral dosage form of claim 22, wherein the acidifier is selected from the group consisting of ascorbic acid, citric acid, fumaric acid, malic acid, maleic acid, malonic acid, aspartic acid, tartaric acid, sorbic acid, succinic acid, glutamic acid, hydrochloride, an acid salt, and combinations thereof.

26. The oral dosage form of claim 22, wherein the carbonic compound is selected from the group consisting of calcium carbonate, dihydroxy aluminum sodium carbonate, magnesium carbonate, potassium bicarbonate, potassium carbonate, sodium bicarbonate, sodium carbonate, zinc carbonate, and combinations thereof.

27. The oral dosage form of claim 22, wherein the water insoluble fluid penetrating agent is selected from the group consisting of sodium starch glycolate, croscarmellose sodium, methylcellulose, carboxy methyl cellulose, microcrystalline cellulose, ion exchange resins, and combinations thereof.

28. The oral dosage form of claim 22, wherein the sustained release polymer is selected from the group consisting of cellulose acetate phthalates, copolymers of methacrylic acid, copolymers of methacrylate, copolymers of methyl methacrylate, ethyl cellulose, hydrogenated glyceryl esters of fatty acids, castro wax, carnauba wax, methacrylic acid ethyl acrylate copolymer, methacrylic acid copolymers with trimethyl ammoniumethyl methacrylate, methcrylic acid-ethyl acrylate copolymer (1:1), neutral polymers of methacrylate, and combinations thereof.

29. The oral dosage form of claim 22, wherein said insoluble hydrophilic insoluble polymer is pregelatinized starch, and said water insoluble fluid penetrating agent is sodium starch glycolate.

30. The oral dosage form of claim 22, wherein said sustained release polymer is a methacrylic acid copolymer.

31. The oral dosage form of claim 22, wherein the basic amine drug in said first gastro-retentive component is different from the basic amine drug in said second gastro-retentive component.

32. The oral dosage form of claim 22, wherein the basic amine drug in said non gastro-retentive component is different from the basic amine drug in said first gastro-retentive component and different from the basic amine drug in said second gastro-retentive component.

33. A tablet for oral administration comprising,
(a) a gastrorentive matrix comprising a basic amine drug, an acidifier,
a carbonic compound, pregeletinized starch, and sodium starch glycolate; and
(b) one or more beads, wherein said bead comprises a core comprising an acidifier, a first layer comprising a basic amine drug, and an outer layer comprising a sustained release polymer,
wherein said one or more beads are embedded in said gastro-retentive matrix.

34. The tablet of claim 33, wherein the acidifier is selected from the group consisting of ascorbic acid, citric acid, fumaric acid, malic acid, maleic acid, malonic acid, aspartic acid, tartaric acid, sorbic acid, succinic acid, glutamic acid, hydrochloride, an acid salt, and combinations thereof.

35. The tablet of claim 33, wherein the carbonic compound is selected from the group consisting of calcium carbonate, dihydroxy aluminum sodium carbonate, magnesium carbonate, potassium bicarbonate, potassium carbonate, sodium bicarbonate, sodium carbonate, zinc carbonate, and combinations thereof.

36. The tablet of claim 33, wherein the sustained release polymer is selected from the group consisting of cellulose acetate phthalates, copolymers of methacrylic acid, copolymers of methacrylate, copolymers of methyl methacrylate, ethyl cellulose, hydrogenated glyceryl esters of fatty acids, castro wax, carnauba wax, methacrylic acid ethyl acrylate copolymer, methacrylic acid copolymers with trimethyl ammoniumethyl methacrylate, methcrylic acid-ethyl acrylate copolymer (1:1), neutral polymers of methacrylate, and combinations thereof.

37. The tablet of claim 33, wherein the basic amine drug in said gastro-retentive matrix is different from the basic amine drug in said bead.

38. A capsule for oral administration comprising,
(a) one or more gastro-retentive beads, wherein said bead comprises a core comprising an acidifier, a first layer comprising a basic amine drug, a carbonic compound, pregeletinized starch, and sodium starch glycolate, and an outer layer comprising a sustained release polymer; and
(b) one or more non gastro-retentive beads, wherein said bead comprises a core comprising an acidifier, and an outer layer comprising a basic amine drug,
wherein said gastro-retentive beads and said non gastro-retentive beads are in a capsule.

39. The capsule of claim 38, wherein the acidifier is selected from the group consisting of ascorbic acid, citric acid, fumaric acid, malic acid, maleic acid, malonic acid, aspartic acid, tartaric acid, sorbic acid, succinic acid, glutamic acid, hydrochloride, an acid salt, and combinations thereof.

40. The capsule of claim 38, wherein the carbonic compound is selected from the group consisting of calcium carbonate, dihydroxy aluminum sodium carbonate, magnesium carbonate, potassium bicarbonate, potassium carbonate, sodium bicarbonate, sodium carbonate, sodium carbonate monohydrate, zinc carbonate, and combinations thereof.

41. The capsule of claim 38, wherein the sustained release polymer is selected from the group consisting of cellulose acetate phthalates, copolymers of methacrylic acid, copolymers of methacrylate, copolymers of methyl methacrylate, ethyl cellulose, hydrogenated glyceryl esters of fatty acids, castro wax, carnauba wax, methacrylic acid ethyl acrylate copolymer, methacrylic acid copolymers with trimethyl ammoniumethyl methacrylate, methcrylic acid-ethyl acrylate copolymer (1:1), neutral polymers of methacrylate, and combinations thereof.

42. The capsule of claim 38, wherein the basic amine drug in said gastro-retentive bead is different from the basic amine drug in said non gastro-retentive bead.

43. A capsule for oral administration comprising,
(a) one or more gastro-retentive tablets, wherein said tablet comprises an acidifier, a basic amine drug, a carbonic compound, pregeletinized starch, and sodium starch glycolate; and
(b) one or more non gastro-retentive beads, wherein said bead comprises a core comprising an acidifier, a first layer on said core, comprising a basic amine drug, and an outer layer comprising a sustained release polymer.
wherein said gastro-retentive tablet and said non gastro-retentive bead are in a capsule.

44. The capsule of claim 43, wherein the acidifier is selected from the group consisting of ascorbic acid, citric acid, fumaric acid, malic acid, maleic acid, malonic acid, aspartic acid, tartaric acid, sorbic acid, succinic acid, glutamic acid, hydrochloride, an acid salt, and combinations thereof.

45. The capsule of claim 43, wherein the carbonic compound is selected from the group consisting of calcium carbonate, dihydroxy aluminum sodium carbonate, magnesium carbonate, potassium bicarbonate, potassium carbonate, sodium bicarbonate, sodium carbonate, sodium zinc carbonate, and combinations thereof.

46. The capsule of claim 43, wherein the sustained release polymer is selected from the group consisting of cellulose acetate phthalates, copolymers of methacrylic acid, copolymers of methacrylate, copolymers of methyl methacrylate, ethyl cellulose, hydrogenated glyceryl esters of fatty acids, castro wax, carnauba wax, methacrylic acid ethyl acrylate copolymer, methacrylic acid copolymers with trimethyl ammoniumethyl methacrylate, methcrylic acid-ethyl acrylate copolymer (1:1), neutral polymers of methacrylate, and combinations thereof.

47. The capsule of claim 43, wherein the basic amine drug in said gastro-retentive tablet is different from the basic amine drug in said non gastro-retentive bead.
